(19) United States
(12) Patent Application Publication
(10) Pub. No.: US 2026/0327110 A1
(43) Pub. Date: Oct. 22, 2026

(54) BACTERIAL COMPOSITION FOR MARINE RESTORATION, MICROBIAL CARRIER, AND USE THEREOF

(71) Applicant: THIRD INSTITUTE OF OCEANOGRAPHY, MNR, Xiamen (CN)

(72) Inventors: Wanpeng Wang, Xiamen (CN); Bin Zhi, Xiamen (CN); Hongxin Chen, Xiamen (CN)

(21) Appl. No.: 19/644,520

(22) Filed: Apr. 10, 2026

(30) Foreign Application Priority Data
Apr. 18, 2025 (CN) 202510490607.0

(51) Int. Cl. C02F 3/10; C02F 3/34; C12N 1/20; C02F 103/08; C12R 1/01

(52) U.S. Cl. CPC C02F 3/10; C02F 3/348; C12N 1/20; C02F 2103/08; C12R 2001/01

(57) ABSTRACT

A bacterial composition for marine restoration, a microbial carrier, and use thereof. A bacterial composition for marine restoration, including an Alcanivorax dieselolei strain B-5 and a Halomonas titanicae strain HT PA16_9 that have viable counts at a ratio of (2-6):1. A microbial carrier, including a carrier and the bacterial composition for marine restoration loaded on the carrier. This microbial carrier efficiently enriches marine microorganisms, promotes microbial community proliferation, increases biomass and biodiversity, and restores seabed environments.

### CROSS REFERENCE TO RELATED APPLICATION

This patent application claims the benefit and priority of Chinese Patent Application No. 202510490607.0 filed with the China National Intellectual Property Administration on Apr. 18, 2025, the disclosure of which is incorporated by reference herein in its entirety as part of the present application.

### TECHNICAL FIELD

The present disclosure belongs to the technical field of microbiology, and particularly relates to a bacterial composition for marine restoration, a microbial carrier, and use thereof.

### BACKGROUND

Marine environmental restoration has become a crucial aspect of marine resource development. Traditional marine restoration primarily relies on physicochemical methods. However, these conventional techniques involve large-scale engineering, high construction costs, potential secondary pollution, and significant environmental impacts. In recent years, biological restoration has emerged as a major alternative. Biological restoration offers advantages such as low cost and high restoration efficiency, typically costing only one-fourth or less of traditional restoration methods. Moreover, biological restoration is environmentally friendly and does not cause secondary pollution, making it a widely recognized approach for environmental restoration. Nevertheless, there are currently limited materials available for biological restoration, and their restoration effects remain suboptimal.

### SUMMARY

In view of this, an objective of the present disclosure is to provide a bacterial composition for marine restoration. A microbial carrier prepared using the bacterial composition for marine restoration efficiently enriches marine microorganisms, promotes microbial community proliferation, increases biomass and biodiversity of the marine microorganisms, and restores seabed environments.

To achieve the above objective, the present disclosure provides the following technical solutions:

The present disclosure provides a bacterial composition for marine restoration, including an Alcanivorax dieselolei strain B-5 and a Halomonas titanicae strain HT PA16_9; where a viable count ratio of the Alcanivorax dieselolei strain B-5 to the Halomonas titanicae strain HT PA16_9 in the bacterial composition for marine restoration is at (2-6):1.

In some embodiments, the viable count ratio of the Alcanivorax dieselolei strain B-5 to the Halomonas titanicae strain HT PA16_9 is at 4:1.

The present disclosure further provides a microbial carrier, including a carrier and the bacterial composition for marine restoration loaded on the carrier.

In some embodiments, the carrier is at least one selected from the group consisting of polyethylene (PE), polypropylene (PP), polyurethane (PU), ceramic, and activated carbon.

The present disclosure further provides a method of preparing the microbial carrier, including: mixing the carrier with a bacterial suspension of the bacterial composition for marine restoration.

In some embodiments, the mixing is conducted at 14°C to 28°C for 1 h to 72 h; and the Alcanivorax dieselolei strain B-5 in the bacterial suspension has an initial concentration of 2×10⁶ CFU/mL to 6×10¹⁰ CFU/mL.

The present disclosure further provides use of the bacterial composition for marine restoration, the microbial carrier, or a microbial carrier prepared by the preparation method in enriching marine microorganisms.

The present disclosure further provides use of the bacterial composition for marine restoration, the microbial carrier, or a microbial carrier prepared by the preparation method in marine restoration.

In some embodiments, the marine restoration includes increasing microbial biomass.

The present disclosure further provides a method for marine restoration, including: immersing the microbial carrier in seawater.

Compared with the prior art, embodiments of the present disclosure have the following advantages:

The present disclosure provides a bacterial composition for marine restoration, including an Alcanivorax dieselolei strain B-5 and a Halomonas titanicae strain HT PA16_9 that have viable counts at a ratio of (2-6):1. By combining the Alcanivorax dieselolei strain B-5 and the Halomonas titanicae strain HT PA16_9 at the specified ratio, the resulting bacterial composition for marine restoration efficiently enriches marine microorganisms, promotes microbial community proliferation, increases biomass, and restores seabed environments.

A microbial carrier is further provided, including a carrier and the bacterial composition for marine restoration loaded on the carrier. This microbial carrier efficiently enriches marine microorganisms, promotes microbial community proliferation, increases biomass and biodiversity, and restores seabed environments. Additionally, the microbial carrier exhibits advantages including low restoration cost, high restoration efficiency, environmental friendliness, and absence of secondary pollution.

The present disclosure further provides use of the bacterial composition for marine restoration, the microbial carrier, or a microbial carrier prepared by the preparation method in enriching marine microorganisms and/or marine restoration. In the examples, using biomass as an indicator, comparisons are made between the microbial carrier, randomly selected microbial carriers, and blank carriers regarding their impacts on seabed microorganisms and marine environments. Results demonstrate that the protein content in the microbial carrier is significantly higher than that in the randomly selected and blank carriers. Moreover, the microbial carrier has enriched diverse microorganisms, with species other than the Halomonas titanicae strain HT PA16_9 and Alcanivorax dieselolei strain B-5 accounting for 59% of the total biomass. This confirms that the method efficiently enriches marine microorganisms, promotes proliferation of marine microbial communities, increases biomass, and achieves marine environmental restoration.

### BRIEF DESCRIPTION OF THE DRAWINGS

FIG. 1 shows biomass measurement results; and

FIG. 2 is a diagram showing the abundance of different microorganisms relative to the total microbial population.

### DETAILED DESCRIPTION OF THE EMBODIMENTS

The present disclosure provides a bacterial composition for marine restoration, including an Alcanivorax dieselolei strain B-5 and a Halomonas titanicae strain HT PA16_9; where

viable counts of the Alcanivorax dieselolei strain B-5 and the Halomonas titanicae strain HT PA16_9 in the bacterial composition for marine restoration are at a ratio of (2-6):1.

In the present disclosure, the Alcanivorax dieselolei strain B-5 is disclosed in the prior art: Li Anzhang; Shao Zongze. Degradation of halogenated compounds by Alcanivorax dieselolei B-5 and its haloalkane dehalogenase DadA [J]. Acta Microbiologica Sinica, 2014, 54(09):1063-1072. DOI:10.13343/j.cnki.wsxb.2014.09.011. The cultivation method for the Alcanivorax dieselolei strain B-5 preferably includes inoculating colonies into 2216E liquid medium. The cultivation is conducted at preferably 22° C to 24° C, more preferably 23° C. The rotational speed during cultivation is preferably 120 rpm to 180 rpm, more preferably 140 rpm to 160 rpm, and most preferably 150 rpm. This cultivation method enhances the activity of Alcanivorax dieselolei strain B-5, thereby effectively enriching marine microorganisms and restoring marine environments.

In the present disclosure, the Halomonas titanicae strain HT PA16_9 is disclosed in Chinese Patent CN110669700B, titled "A high-efficiency petroleum hydrocarbon-degrading bacterium PA16_9, its screening method and applications". The cultivation method for the Halomonas titanicae strain HT PA16_9 is preferably identical to that of the Alcanivorax dieselolei strain B-5 described above, and thus is not reiterated herein. This method enhances the activity of Halomonas titanicae strain HT PA16_9, contributing to effective enrichment of marine microorganisms and marine environment restoration.

In the present disclosure, the viable counts of the Alcanivorax dieselolei strain B-5 and the Halomonas titanicae strain HT PA16_9 are at a ratio of preferably 4:1. This ratio of viable counts facilitates stable long-term colonization of the bacterial composition for marine restoration, enhancing its efficacy in enriching marine microorganisms and restoring marine environments. Experimental results demonstrate that combining the Alcanivorax dieselolei strain B-5 and the Halomonas titanicae strain HT PA16_9 at the specified ratio yields a composition that efficiently enriches marine microorganisms, promotes microbial community proliferation, increases biomass, and restores seabed environments.

The present disclosure further provides a microbial carrier, including a carrier and the bacterial composition for marine restoration loaded on the carrier.

In the present disclosure, the carrier is preferably at least one selected from the group consisting of PE, PP, PU, ceramic, and activated carbon, more preferably includes a Moving Bed Biofilm Reactor (MBBR) media. This carrier not only provides a suitable growth environment for microorganisms to enhance marine microorganism enrichment efficiency and restore marine environments, but also facilitates quantification of the restoration efficacy of the bacterial composition for marine restoration. In the examples, K6 biological media is used as the carrier, where K6 is purchased from Henan Jiekang Environmental Technology Co., Ltd. under the product name Fluidized Bed MBBR Media K6.

The present disclosure further provides a method of preparing the microbial carrier, including: mixing the carrier with a bacterial suspension of the bacterial composition for marine restoration.

In the present disclosure, the initial concentration of Alcanivorax dieselolei strain B-5 in the bacterial suspension is preferably 2×10⁶ CFU/mL to 6×10¹⁰ CFU/mL, more preferably 2×10⁷ CFU/mL to 6×10⁹ CFU/mL, further preferably 2×10⁸ CFU/mL to 6×10⁸ CFU/mL, and most preferably 4×10⁸ CFU/mL. The initial concentration of Halomonas titanicae strain HT PA16_9 in the bacterial suspension is preferably 0.5×10⁶ to 1.5×10¹⁰ CFU/mL, more preferably 0.5×10⁷ CFU/mL to 1.5×10⁹ CFU/mL, further preferably 0.8×10⁸ CFU/mL to 1.2×10⁸ CFU/mL, and most preferably 1×10⁸ CFU/mL. The mixing is conducted for preferably 1 h to 72 h, more preferably 2 h to 48 h, further preferably 3 h to 12 h, and most preferably 4 h at preferably 14°C to 28°C, more preferably 18°C to 26°C, and optimally 23°C. Agitation is applied during mixing at a rotational speed preferably of 120 rpm to 180 rpm, more preferably 140 rpm to 160 rpm, and most preferably 150 rpm. The carrier-to-suspension ratio by volume is preferably (1-2):(3-6), more preferably 2:3. After the mixing, the strains in the bacterial composition for marine restoration adhere to and colonize on the carrier, yielding the microbial carrier. This carrier provides abundant attachment sites and a stable growth environment for both the bacterial composition for marine restoration's strains and other marine microorganisms, enhancing microbial density and activity per unit volume. Following adhesion to the carrier, the strains in bacterial composition for marine restoration establish a stable microenvironment, which maximizes the activity in enriching marine microorganisms and restoring marine environments. The microbial carrier prepared by this preparation method significantly improves marine microorganism enrichment efficacy, increases microbial biomass, and restores seabed environments.

The present disclosure further provides use of the bacterial composition for marine restoration, the microbial carrier, or a microbial carrier prepared by the preparation method in enriching marine microorganisms.

The present disclosure further provides use of the bacterial composition for marine restoration, the microbial carrier, or a microbial carrier prepared by the preparation method in marine restoration.

In the present disclosure, the marine restoration preferably includes increasing microbial biomass. The marine restoration further preferably includes enhancing microbial diversity. The term "marine" preferably refers to seawater bodies where microbial diversity and biomass have been reduced due to various engineering activities.

The present disclosure further provides a method for marine restoration, including: immersing the microbial carrier in seawater.

In the present disclosure, regarding methods for immersing the microbial carrier in seawater, the immersion depth or deployment density are not specifically restricted. These parameters may be determined using conventional methods in the art or according to practical requirements. In an optional embodiment, the preferred immersion method includes mounting the microbial carrier on a polyvinyl chloride (PVC) mesh with the carrier surface facing upward, submerged in the seawater body. The distance between the submerged microbial carrier and the water surface is preferably 2.5 m to 3.5 m, more preferably 2.8 m to 3.2 m, and most preferably 3 m. The PVC mesh dimensions are preferably 30 cm × 30 cm × 0.6 cm. The seawater is preferably nutrient-deprived and/or contaminated by resource extraction activities. The immersion of microbial carrier is conducted for preferably greater than 7 days, more preferably 7 days to 50 days, further preferably 10 days to 40 days, and most preferably 28 days. This method effectively activates the microbial carrier, enhances marine microorganism enrichment efficiency, improves marine microbial diversity, and restores marine environments.

The present disclosure provides a method for enriching marine microorganisms, which is preferably identical to the aforementioned method for marine restoration and thus not reiterated herein.

In the examples, comparisons are made between the microbial carrier, randomly selected microbial carriers, and blank carriers regarding their impacts on seabed microorganisms and marine environments. Results demonstrate that the protein content in the microbial carrier is significantly higher than that in the randomly selected and blank carriers. Moreover, the microbial carrier has enriched diverse microorganisms, with species other than the Halomonas titanicae strain HT PA16_9 and Alcanivorax dieselolei strain B-5 accounting for 59% of the total biomass. These findings confirm the method's efficacy in enriching marine microorganisms, promoting microbial community proliferation, and increasing biomass. Furthermore, prior art (Sitthi, A., Pimple, U., Piponiot, C. et al. Assessing the effectiveness of mangrove rehabilitation using above-ground biomass and structural diversity. Sci Rep 15, 7839 (2025). doi.org/10.1038/s41598-025-92514-7) establishes biomass as a critical indicator of marine environmental recovery. Therefore, this method effectively restores seabed environments.

In order to further illustrate the present disclosure, the technical solutions of the present disclosure are described in detail below in connection with accompanying drawings and examples, but these examples should not be understood as a limit to the protection scope of the present disclosure.

### Example 1

### 1. Preparation of bacterial composition for marine restoration

Alcanivorax dieselolei strain B-5 and Halomonas titanicae strain HT PA16_9 were separately inoculated at 1:100 ratio from 10⁴ CFU/mL bacterial suspensions into 2216E liquid medium. The cultivation proceeded at 23° C and 150 rpm, yielding PA16_9 bacterial suspension and B-5 bacterial suspension.

The PA16_9 and B-5 bacterial suspensions were then mixed to achieve final concentrations of 4×10⁸ CFU/mL for Alcanivorax dieselolei strain B-5 and 1×10⁸ CFU/mL for Halomonas titanicae strain HT PA16_9. The resulting bacterial suspension mixture was designated as the bacterial composition for marine restoration.

### 2. Preparation of Microbial Carrier

K6 high-density polyethylene (HDPE) media was combined with the bacterial composition for marine restoration at a 2:3 volume ratio, ensuring complete submersion of the K6 HDPE media. The mixture underwent agitated cultivation at 23° C and 150 rpm in a rotary shaker for 4 h. This produced carrier loaded with the bacterial composition for marine restoration, which was subsequently labeled as the microbial carrier.

### Comparative Example 1

### Preparation Method of Random Microbial Carrier

Escherichia coli isolated from seawater served as a random bacterial agent. A suspension of this strain at 10⁴ CFU/mL was inoculated at 1:100 ratio into 2216E liquid medium and cultivated at 23° C and 150 rpm to obtain a random bacterial suspension.

K6 HDPE media was then combined with this random bacterial suspension (concentration: 5×10⁸ CFU/mL) at a 2:3 volume ratio, and complete submersion of the carrier was ensured. The mixture underwent agitated cultivation at 23°C and 150 rpm in a rotary shaker for 4 h. This yielded a carrier loaded with the random bacterial agent, which was designated as the random microbial carrier.

### Example 2

### Use of Microbial Carrier in Seabed restoration

The microbial carrier prepared in Example 1, random microbial carrier from Comparative Example 1, and blank carrier (K6 HDPE media without bacterial agent) were uniformly mounted on PVC meshes (30 cm × 30 cm × 0.6 cm) at 100 particles per panel. The PVC meshes were suspended at a dock, and submerged approximately 3 meters below the seawater surface. Every 7 days, the microbial carrier and the random microbial carrier were retrieved for biomass measurement until day 28. The biomass referred to protein quantity per cubic centimeter of carrier, while biological composition indicated relative abundance of attached organisms. The retrieved microbial carriers, random microbial carriers, and blank carriers were rinsed with sterilized seawater, and their biomasses were determined, respectively.

Biomass quantification: The carriers underwent ultrasonic disruption (ice bath, 30 W, 3 s pulse/10 s interval, 30 cycles) in PBS lysis buffer containing 8% lysozyme and 10% Triton X-100. The resulting lysates were filtered, and then centrifuged at 12,000 rpm (4°C, 10 min). The supernatant (1 ml) was mixed with 1 mL Bradford reagent, incubated at room temperature for 30 min, and measured for absorbance at 590 nm. The protein concentration was calculated against a standard curve.

Biological composition analysis: genomic DNA was extracted using Fast DNA™ Spin Kit for Soil (MP Bio). The samples were homogenized with 978 μL Sodium Phosphate Buffer and 122 μL MgCl2-tris (MT) Buffer, vortexed for 40 s, and centrifuged at 14,000×g for 5 min. A resulting supernatant was mixed with 250 μL protein precipitation solution (PPS), combined with 1 mL Binding Matrix, inverted manually, and incubated for 3 min. After removal of 500 μL supernatant, the pellets were resuspended and transferred to SPIN™ Filter, followed by centrifugation at 14,000×g for 1 min. The flow-through was discarded, and 500 μL SEWS-M solution was added. After centrifugation at 14,000 × g for 1 min, the flow-through was discarded. SPIN™ Filter was centrifuged at 14,000×g for 2 min, collection tubes were replaced, and filters stood at room temperature for 5 min. Finally, 50 μL sterile water was added to SPIN™ Filter and centrifuged at 14,000×g for 1 min to elute DNA into collection tubes. The samples was subjected to 16S diversity sequencing by Sangon Biotech (Shanghai) Co., Ltd. for microbial composition analysis.

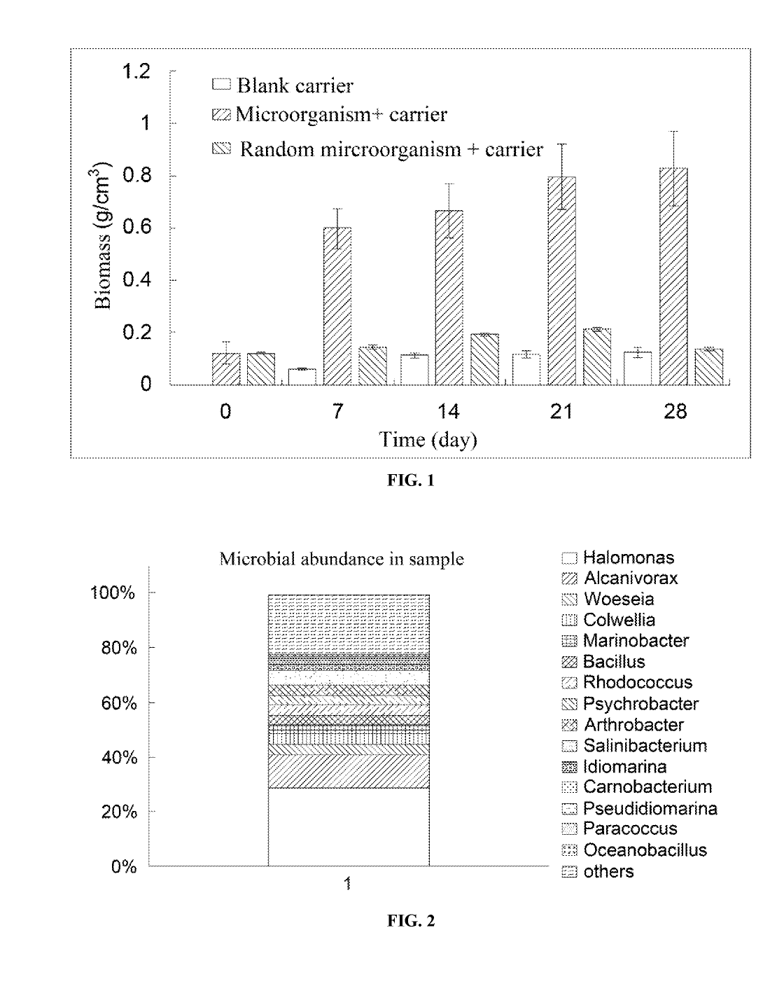

Biomass measurement results are shown in FIG. 1, while the proportions of different microorganisms in the microbial carrier from Example 1 relative to total microbial weight are presented in FIG. 2. FIG. 1 demonstrated significantly higher biomass in the microbial carrier compared to both that of the blank carrier and that of the random microbial carrier. FIG. 2 revealed the enrichment of diverse microorganisms in the microbial carrier, with species other than Halomonas titanicae strain HT PA16_9 and Alcanivorax dieselolei strain B-5 accounting for 59% of total biomass. This confirmed the carrier's efficacy in efficiently enriching marine microorganisms, promoting microbial community proliferation, and increasing biomass. As prior art establishes biomass as a critical indicator of marine environmental recovery (Sitthi, A., Pimple, U., Piponiot, C. et al. Assessing the effectiveness of mangrove rehabilitation using above-ground biomass and structural diversity. Sci Rep 15, 7839 (2025).doi.org/10.1038/s41598-025-92514-7), the microbial carrier effectively restores seabed environments.

Although the present disclosure is described in detail in conjunction with the foregoing examples, they are only a part of, not all of, the embodiments of the present disclosure. Other embodiments can be obtained based on these examples without creative efforts, and all of these embodiments shall fall within the protection scope of the present disclosure.

What is claimed is:

1. A bacterial composition for marine restoration, comprising an Alcanivorax dieselolei strain B-5 and a Halomonas titanicae strain HT PA16_9; wherein a viable count ratio of the Alcanivorax dieselolei strain B-5 to the Halomonas titanicae strain HT PA16_9 in the bacterial composition for marine restoration is at 2-6:1.

2. The bacterial composition for marine restoration of claim 1, wherein the viable count ratio of the Alcanivorax dieselolei strain B-5 to the Halomonas titanicae strain HT PA16_9 is at 4:1.

3. A microbial carrier, comprising a carrier and the bacterial composition for marine restoration of claim 1, wherein the bacterial composition for marine restoration is loaded on the carrier.

4. The microbial carrier of claim 3, wherein the viable count ratio of the Alcanivorax dieselolei strain B-5 to the Halomonas titanicae strain HT PA16_9 is at 4:1.

5. The microbial carrier of claim 3, wherein the carrier is at least one selected from the group consisting of polyethylene (PE), polypropylene (PP), polyurethane (PU), ceramic, and activated carbon.

6. A method of preparing the microbial carrier of claim 3, comprising: mixing the carrier with a bacterial suspension of the bacterial composition for marine restoration comprising an Alcanivorax dieselolei strain B-5 and a Halomonas titanicae strain HT PA16_9; wherein a viable count ratio of the Alcanivorax dieselolei strain B-5 to the Halomonas titanicae strain HT PA16_9 in the bacterial composition for marine restoration is at 2-6:1.

7. The method of claim 6, wherein the carrier is at least one selected from the group consisting of polyethylene (PE), polypropylene (PP), polyurethane (PU), ceramic, and activated carbon.

8. The method of claim 6, wherein the mixing is conducted at 14°C to 28°C for 1 hour to 72 hours; and the Alcanivorax dieselolei strain B-5 in the bacterial suspension has an initial concentration of 2×106 colony-forming unit (CFU)/mL to 6×1010 CFU/mL.

9. A method for enriching marine microorganisms, comprising applying to seawater the bacterial composition for marine restoration of claim 1.

10. The method of claim 9, wherein the viable count ratio of the Alcanivorax dieselolei strain B-5 to the Halomonas titanicae strain HT PA16_9 is at 4:1

11. A method for enriching marine microorganisms, comprising immersing the microbial carrier of claim 3 in seawater.

12. The method of claim 11, wherein the carrier is at least one selected from the group consisting of polyethylene (PE), polypropylene (PP), polyurethane (PU), ceramic, and activated carbon.

13. A method of marine restoration, comprising applying to seawater the bacterial composition for marine restoration of claim 1.

14. The method of claim 13, wherein the viable count ratio of the Alcanivorax dieselolei strain B-5 to the Halomonas titanicae strain HT PA16_9 is at 4:1

15. The method of claim 13, wherein the marine restoration comprises increasing microbial biomass.

16. A method for marine restoration, comprising: immersing the microbial carrier of claim 3 in seawater.

17. The method of claim 15, wherein the carrier is at least one selected from the group consisting of polyethylene (PE), polypropylene (PP), polyurethane (PU), ceramic, and activated carbon.
